(19) United States
(12) Patent Application Publication
(10) Pub. No.: US 2012/0295267 A1
(43) Pub. Date: Nov. 22, 2012

(54) Detecting DNA Mismatch Repair-Deficient Colorectal Cancers

(75) Inventors: Ajay Goel, Dallas, TX (US); C. Richard Boland, Dallas, TX (US); Takeshi Nagasaka, Okayama (JP)

(73) Assignee: Baylor Research Institute, Dallas, TX (US)

(21) Appl. No.: 13/465,668

(22) Filed: May 7, 2012

Related U.S. Application Data
(60) Provisional application No. 61/486,610, filed on May 16, 2011.

(51) Int. Cl. C12Q 1/68; C12M 1/40

(52) U.S. Cl. 435/6.12; 435/287.2

(57) ABSTRACT

Use of a CAT25 mononucleotide marker in a novel tetraplex PCR for the detection of MSH6-defective colorectal cancers (CRCs) is described herein. The tetraplex PCR of the present invention offers a facile, robust, less expensive (compared to the original pentaplex assay), highly sensitive, and specific assay for the identification of microsatellite instability (MSI) in CRCs.

### CROSS-REFERENCE TO RELATED APPLICATIONS

This application claims priority to U.S. Provisional Application Ser. No. 61/486,610, filed May 16, 2011, the entire contents of each are incorporated herein by reference.

### STATEMENT OF FEDERALLY FUNDED RESEARCH

This invention was made with U.S. Government support under Contract No. R01 CA72851 awarded by the National Institutes of Health (NIH). The government has certain rights in this invention.

### TECHNICAL FIELD OF THE INVENTION

The present invention relates in general to colorectal cancer (CRC) detection methods and techniques, and more particularly, to tetraplex PCR for the detection of microsatellite instability (MSI)-positive, and in particular, MSH6-deficient CRCs.

### REFERENCE TO A SEQUENCE LISTING

None.

### BACKGROUND OF THE INVENTION

Without limiting the scope of the invention, its background is described in connection with marker panels and screening strategies for colorectal cancer (CRC) detection and diagnosis and more particularly for microsatellite instability (MSI)-positive CRCs.

U.S. Patent Publication No. 20090317815 (Hamelin and Suraweera, 2009) relates to a method for evaluating microsatellite instability associated with a tumour, which entails the steps of amplifying microsatellite loci in a biological sample containing genomic DNA from the tumour and determining sizes of DNA amplification products, wherein at least one microsatellite locus selected from the group consisting of NR 21, NR 22, NR 24 and NR 27, is amplified. The method further comprises at least one pair of primers selected from the group consisting of: a) a pair of primers for amplifying microsatellite locus BAT-25; and b) a pair of primers for amplifying microsatellite locus BAT-26.

U.S. Pat. No. 7,662,595 issued to Findeisen et al. (2010) provides a method for assessment of the Microsatellite Instability (MSI) status of medically relevant conditions associated with MSI phenotype such as e.g. neoplastic lesions. The method is based on the analysis of a monomorphic T25 (CAT25) mononucleotide repeat located in the 3′-UTR of the Caspase 2 (CASP2) gene. Based on the determination of the length of the named mononucleotide repeat the presence or absence of MSI may be assessed. Determination of the length is performed in a single PCR procedure. Alternatively an enhanced assessment could be performed by combining the CAT25 marker with further markers such as BAT25 and BAT26 in a single multiplex PCR process. The one or more microsatellite markers are selected from the group consisting of D2S123, D17S250, D5S346, BAT25, BAT26, BAT40, APdelta3, U79260, PPP3CA, CTNNB1, GTF2E1, NR-21, NR-22, NR-24 and Mono27.

### SUMMARY OF THE INVENTION

A tetraplex PCR for detecting microsatellite instability (MSI)-positive CRCs, and in particular, MSH6-defective colorectal cancers (CRCs) is disclosed in various embodiments of the present invention. The present invention further describes the robustness of CAT25 mononucleotide marker for the identification of MSH6-deficient CRCs.

In one embodiment the instant invention discloses a system for detecting microsatellite instability (MSI) in a biological sample from a human or animal subject comprising a sufficient number of mononucleotide markers to allow for a detection of a change in a MSH6 gene expression, MSH6 protein expression, or both, wherein the mononucleotide markers comprise CAT25, BAT25, BAT26, NR21, NR22, NR24, NR27, and any combinations thereof. The biological sample used in the system of the instant invention is a sample isolated from a tumor selected from the group consisting of colorectal cancers, ovarian cancer, urothelial cancers, colorectal tract tumors, colorectal carcinoma, colorectal adenoma, colorectal polyps, gastrointestinal tract tumors, small intestine carcinomas, small intestine polyps, endometrial tumors, endometrial carcinoma, endometrial hyperplasia, and any combinations thereof. In a specific aspect of the system disclosed hereinabove the tumor is an MSH6-deficient colorectal cancer. In another aspect the PCR system comprises CAT25, BAT26, NR21, and NR27. In yet another aspect the biological sample is selected from the group consisting of a tissue sample, a fecal sample, a cell homogenate, and one or more biological fluids.

Another embodiment of the instant invention provides a method for detecting one or more tumors caused by microsatellite instability (MSI) in a human or animal subject comprising the steps of: (i) obtaining a biological sample from the human or the animal subject, wherein the tissue or the biological sample is a sample isolated from a tumor selected from the group consisting of colorectal cancers, ovarian cancer, urothelial cancers, colorectal tract tumors, colorectal carcinoma, colorectal adenoma, colorectal polyps, gastrointestinal tract tumors, small intestine carcinomas, small intestine polyps, endometrial tumors, endometrial carcinoma, endometrial hyperplasia, and any combinations thereof, (ii) isolating a DNA from the biological sample, (iii) amplifying the DNA in a PCR system comprising a sufficient number of mononucleotide markers to allow for a detection of a change in a MSH6 gene expression, MSH6 protein expression, or both, wherein the mononucleotide markers comprise CAT25, BAT25, BAT26, NR21, NR22, NR24, NR27, and any combinations thereof, and (iv) detecting the presence of one or more tumors caused by MSI based on an expression of one or more mismatch repair (MMR) genes, wherein the MMR genes are selected from the group consisting of MLH1, MSH2, MSH3, MSH6, and PMS2.

The method as disclosed hereinabove further comprises the optional steps of: a) obtaining a biological sample from a healthy human or animal subject, wherein the healthy human or animal subject is further defined as a subject not suffering from one or more tumors caused by MSI, b) isolating a DNA from the biological sample of the healthy human or animal subject, c) amplifying the DNA in the PCR system, and d) comparing the expression of the one or more MMR genes in the biological sample of the healthy human or animal subject with the expression in the biological sample of the human or animal subject suspected of having the one or more tumors caused by MSI.

In one aspect of the method the biological sample is selected from the group consisting of a tissue sample, a fecal sample, a cell homogenate, and one or more biological fluids. In another aspect of the method a decrease or an inactivation in the expression of the one or more MMR genes is indicative of the one or more tumors caused by MSI. In specific aspects of the method the tumor is a MSH6-deficient colorectal cancer and the PCR system comprises CAT25, BAT26, NR21, and NR27.

Yet another embodiment of the instant invention discloses a polymerase chain reaction (PCR) system for detection of a MSH6-deficient colorectal cancer (CRC) comprising: a CAT25 mononucleotide marker; and at least one other marker selected from the group consisting of BAT25, BAT26, NR21, NR22, NR24, NR27. In a specific aspect the PCR system disclosed herein, comprises CAT25, BAT26, NR21, and NR27. In a related aspect the system is capable of detecting 85-100% of all MSH6-deficient CRCs. In another aspect the system detects 85%, 86%, 87%, 88%, 89%, 90%, 91%, 92%, 93%, 94%, 95%, 96%, 97%, 98%, 99%, and 100% of all MSH6-deficient CRCs.

The present invention in one embodiment relates to a method for detecting MSH6-deficient colorectal cancers (CRCs) in a human subject comprising the steps of: identifying the human subject suspected of having the MSH6-deficient CRC, obtaining a tissue sample from the human subject, isolating a DNA from the tissue sample, amplifying the DNA in a PCR system comprising: (i) a CAT25 mononucleotide marker and (ii) at least one other marker selected from the group consisting of BAT25, BAT26, NR21, NR22, NR24, NR27, and detecting the presence of the MSH6-deficient CRC based on a reduced expression or an inactivation of the MSH6 gene, MSH6 protein on, or both in the tissue sample of the human subject suspected of having the MSH6-deficient CRC.

The method disclosed above further comprises the optional steps of: obtaining a tissue sample from a healthy human subject, wherein the healthy human subject is further defined as a subject not suffering from one or more tumors caused by microsatellite instability (MSI), isolating a DNA from the tissue sample of the healthy human subject, amplifying the DNA in the PCR system, and comparing a MSH6 gene expression, MSH6 protein expression or both in the tissue sample of the healthy human subject with the MSH6 gene expression, MSH6 protein expression, or both in the tissue sample of the human subject suspected of having the MSH6-deficient CRC.

In one aspect the PCR system comprises CAT25, BAT26, NR21, and NR27. In another aspect the system is capable of detecting 85-100% of all MSH6-deficient CRCs. In yet another aspect the system detects 85%, 86%, 87%, 88%, 89%, 90%, 91%, 92%, 93%, 94%, 95%, 96%, 97%, 98%, 99%, and 100% of all MSH6-deficient CRCs.

Another embodiment of the present invention discloses a tetraplex PCR system for detection of MSH6-deficient colorectal cancer (CRC) comprising a CAT25 mononucleotide marker, a BAT26 mononucleotide marker, a NR21 mononucleotide marker, and a NR27 mononucleotide marker. In one aspect the system is capable of detecting 85-100% of all MSH6-deficient CRCs.

In yet another embodiment the present invention discloses a method for detecting MSH6-deficient colorectal cancers (CRCs) in a human subject comprising the steps of: i) identifying the human subject suspected of having the MSH6-deficient CRC, ii) obtaining a tissue sample from the human subject, iii) isolating a genomic DNA from the tissue sample, iv) amplifying the genomic DNA in a tetraplex PCR system comprising a CAT25 mononucleotide marker, a BAT26 mononucleotide marker, a NR21 mononucleotide marker and a NR27 mononucleotide marker, and v) detecting the presence of the MSH6-deficient CRC based on a reduced expression or an inactivation of the MSH6 gene, protein, or both in the tissue or the biological sample of the human subject suspected of having the MSH6-deficient CRC.

The method disclosed above further comprises the optional steps of: obtaining a tissue sample from a healthy human subject, wherein the healthy human subject is further defined as a subject not suffering from one or more tumors caused by microsatellite instability (MSI), isolating a genomic DNA from the tissue sample of the healthy human subject; amplifying the genomic DNA in the PCR system; and comparing a MSH6 gene expression, MSH6 protein expression, or both in the tissue sample of the healthy human subject with the MSH6 gene expression, MSH6 protein expression, or both in the tissue or the biological sample of the human subject suspected of having the MSH6- deficient CRC. In one aspect the system is capable of detecting 85-100% of all MSH6-deficient CRCs.

The present invention also provides a kit for the detection of a microsatellite instability (MSI) in a tissue or a biological sample of a human subject suspected of having colorectal cancer (CRC) comprising: a CAT25 mononucleotide marker; at least one other marker selected from the group consisting of BAT25, BAT26, NR21, NR22, NR24, NR27; and instructions for use of the kit in the detection of the MSI in the tissue or the biological sample of the human subject suspected of having the colorectal cancer. In one aspect the kit detects a decreased expression, an inactivation, or both of one or more mismatch repair (MMR) genes in the tissue or the biological sample of the human subject, wherein the MMR genes comprise MLH1, MSH2, MSH3, MSH6, and PMS2. In a specific aspect the MMR gene is MSH6. In another aspect the kit is capable of detecting 85-100% of all MSH6-deficient CRCs. In yet another aspect the kit comprises CAT25, BAT26, NR21, and NR27.

Another embodiment of the instant invention discloses a kit comprising one or more pairs of primers for amplifying at least four microsatellite loci selected from the group consisting of CAT25, BAT25, BAT26, NR21, NR22, NR24, NR27, and any combinations thereof. In one aspect the one or more pairs of primers amplify CAT25, BAT26, NR21, and NR27. In another aspect the kit is adapted for use in the detection of a microsatellite instability (MSI) colorectal cancer (CRC) from a tissue or a biological sample of a human subject, wherein the CRC is a MSH6-deficient CRC.

The present invention in one embodiment provides a kit for detecting MSH6-deficient colorectal cancer (CRC) in a human subject comprising one or more pairs of primers for amplifying four microsatellite loci comprising CAT25, BAT26, NR21, and NR27.

Another embodiment of the instant invention describes a system for detecting microsatellite instability (MSI) in a biological sample from a human or animal subject consisting essentially of at least four mononucleotide markers, wherein the mononucleotide markers comprise CAT25, BAT25, BAT26, NR21, NR22, NR24, NR27, and any combinations thereof, wherein the four mononucleotide markers are capable of detecting a change in a MSH6 gene expression, MSH6 protein expression, or both. In one aspect the biological sample is a sample isolated from a tumor selected from the group consisting of colorectal cancers, ovarian cancer, urothelial cancers, colorectal tract tumors, colorectal carcinoma, colorectal adenoma, colorectal polyps, gastrointestinal tract tumors, small intestine carcinomas, small intestine polyps, endometrial tumors, endometrial carcinoma, endometrial hyperplasia, and any combinations thereof. In specific aspects of the system the tumor is an MSH6-deficient colorectal cancer and the system detects CAT25, BAT26, NR21, and NR27. In one aspect the system comprises detection using polymerase chain reaction (PCR). In yet another aspect the biological sample is selected from the group consisting of a tissue sample, a fecal sample, a cell homogenate, and one or more biological fluids.

### BRIEF DESCRIPTION OF THE DRAWINGS

None.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “colorectal cancer” includes the well-accepted medical definition that defines colorectal cancer as a medical condition characterized by cancer of cells of the intestinal tract below the small intestine (i.e., the large intestine (colon), including the cecum, ascending colon, transverse colon, descending colon, sigmoid colon, and rectum). Additionally, as used herein, the term “colorectal cancer” also further includes medical conditions, which are characterized by cancer of cells of the duodenum and small intestine (jejunum and ileum).

The term “tissue”, “tissue sample” and “biological sample” should be understood to include any material composed of fluids or one or more cells, either individual or in complex with any matrix or in association with any chemical that can be sampled, e.g., a portion or sub portion can be isolated, contacted or extracted. The definition shall include any biological or organic material and any cellular subportion, product or by-product thereof. The definition of “tissue”, “tissue sample” and “biological sample” should be understood to include without limitation body fluids, including secretions, excretions, interstitial fluids, blood, plasma, exhalations, breath, and any body tissue including, without limitation sperm, eggs, and embryos. Also included within the definition of “tissue” or “biological sample” for purposes of this invention are certain defined acellular structures such as dermal layers of skin that have a cellular origin but are no longer characterized as cellular or cells and cellular debris obtained from, e.g., sweat and stool. The term “stool” as used herein is a clinical term that refers to feces excreted by humans.

The term “gene” as used herein refers to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated. The term “allele” or “allelic form” refers to an alternative version of a gene encoding the same functional protein but containing differences in nucleotide sequence relative to another version of the same gene.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally occurring nucleotides (such as DNA and RNA), or analogs of naturally occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally occurring or modified nucleic acid bases attached to a polyamide backbone. Nucleic acids can be either single stranded or double stranded.

The term “polymerase chain reaction” (PCR) refers to the method of K. B. Mullis U.S. Pat. Nos. 4,683,195, 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as DCTP or DATP, into the amplified segment). In addition to genomic DNA, any oligonucleotide sequence can be amplified with the appropriate set of primer molecules. In particular the amplified segments created by the PCR process itself are, themselves, efficient templates for subsequent PCR amplifications.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2nd Ed. Cold Spring Harbor Press (1989) which is hereby incorporated by reference in its entirety for all purposes above.

The term “biomarker” as used herein in various embodiments refers to a specific biochemical in the body that has a particular molecular feature to make it useful for diagnosing and measuring the progress of disease or the effects of treatment. For example, common metabolites or biomarkers found in a person's breath, and the respective diagnostic condition of the person providing such metabolite include, but are not limited to, acetaldehyde (source: ethanol, X-threonine; diagnosis: intoxication), acetone (source: acetoacetate; diagnosis: diet/diabetes), ammonia (source: deamination of amino acids; diagnosis: uremia and liver disease), CO (carbon monoxide) (source: CH₂Cl₂ , elevated % COHb; diagnosis: indoor air pollution), chloroform (source: halogenated compounds), dichlorobenzene (source: halogenated compounds), diethylamine (source: choline; diagnosis: intestinal bacterial overgrowth), H (hydrogen) (source: intestines; diagnosis: lactose intolerance), isoprene (source: fatty acid; diagnosis: metabolic stress), methanethiol (source: methionine; diagnosis: intestinal bacterial overgrowth), methylethylketone (source: fatty acid; diagnosis: indoor air pollution/diet), O-toluidine (source: carcinoma metabolite; diagnosis: bronchogenic carcinoma), pentane sulfides and sulfides (source: lipid peroxidation; diagnosis: myocardial infarction), H2S (source: metabolism; diagnosis: periodontal disease/ovulation), MeS (source: metabolism; diagnosis: cirrhosis), and Me₂S (source: infection; diagnosis: trench mouth).

As used herein the term “immunohistochemistry (IHC)” also known as “immunocytochemistry (ICC)” when applied to cells refers to a tool in diagnostic pathology, wherein panels of monoclonal antibodies can be used in the differential diagnosis of undifferentiated neoplasms (e.g., to distinguish lymphomas, carcinomas, and sarcomas) to reveal markers specific for certain tumor types and other diseases, to diagnose and phenotype malignant lymphomas and to demonstrate the presence of viral antigens, oncoproteins, hormone receptors, and proliferation-associated nuclear proteins.

The term “statistically significant” differences between the groups studied, relates to condition when using the appropriate statistical analysis (e.g. Chi-square test, t-test) the probability of the groups being the same is less than 5%, e.g. p<0.05. In other words, the probability of obtaining the same results on a completely random basis is less than 5 out of 100 attempts.

The term “candidate drug” as used herein refers to any compound, of whatever origin, suitable for being screened for its activity in reducing the number of colorectal cells that have decreased MSH3 expression according to the methods of the present invention.

The term “genotoxic agent” as used herein is defined to include both chemical and physical agents capable of causing damage to human DNA or the gene. Carcinogens and mutagens are common examples of chemical genotoxic agents, while UV radiation, γand X-rays and the like when they produce oxidized DNA product are common examples of physical genotoxic agents.

The term “anti-neoplastic agent” refers to agents that have the functional property of inhibiting the development or progression of a neoplasm in a mammal, e.g., a human, and may also refer to the inhibition of metastasis or metastatic potential.

The term “kit” or “testing kit” denotes combinations of reagents and adjuvants required for an analysis. Although a test kit consists in most cases of several units, one-piece analysis elements are also available, which must likewise be regarded as testing kits.

The present invention describes a novel tetraplex PCR for the detection of MSH6-defective colorectal cancers (CRCs). The present inventors compared the performance of the novel tetraplex PCR with the pentaplex PCR in its ability to detect microsatellite instability (MSI)-positive, and in particular, MSH6-deficient CRCs. The present invention further describes the robustness of CAT25 mononucleotide marker for the identification of MSH6-deficient CRCs.

MSI is defined as the accumulation of insertion-deletion mutations at short repetitive DNA sequences (or ‘microsatellites’) is a characteristic feature of cancer cells with DNA mismatch repair (MMR) deficiency. Inactivation of any of several MMR genes, including MLH1, MSH2, MSH6 and PMS2, can result in MSI. Originally, MSI was shown to correlate with germline defects in MMR genes in patients with Lynch syndrome (LS), where >90% of colorectal cancer (CRC) patients exhibit MSI. It was later recognized that MSI also occurs in ˜12% of sporadic CRCs occurring in patients that lack germline MMR mutations, and MSI in these patients is due to promoter methylation-induced silencing of the MLH1 gene expression. Determination of MSI status in CRC has clinical use for identifying patients with germline defects predisposing to MMR-deficiency. Additionally, MSI status has prognostic and therapeutic implications, because MSI CRCs typically have a better prognosis, and these cancers are less responsive to 5FU-based adjuvant chemotherapy.

Since its initial discovery more than a decade ago, the methods and criteria to determine MSI in CRC have constantly evolved. However, there is still a lack of consensus on the use of various MSI assays that are more robust, inexpensive and would result in MSI analyses that best represents MMR-deficiency in laboratories worldwide. In an effort to unify MSI analysis in CRC, in 1997 an National Cancer Institute (NCI) workshop recommended using a reference panel of five MSI markers that consisted of 2 mononucleotide repeat markers (BAT26 and BAT25) and 3 dinucleotide repeat markers (D2S123, D5S346 and D17S250). In a follow-up NCI workshop, the panel recognized some of the limitations of the original markers, primarily due to the inclusion of the 3 dinucleotide markers. First, it was recognized that the dinucleotide 74repeat markers were more suitable for identifying MSI-L tumors, while mononucleotide repeat markers were more specific and sensitive for the determination of MSI (or MSI-H) CRCs. Second, due to the polymorphic nature of dinucleotide markers, these required the availability of not just tumor but matching normal DNA from each individual to interpret MSI results.

More recently work by the present inventors and others have shown that a panel of five quasi-monomorphic mononucleotide repeat markers (BAT25, BAT26, NR21, NR24 and NR27), in a pentaplex PCR obviates the need for normal DNA from each CRC patient, and may offer better specificity and sensitivity than the NCI-panel markers (Goel et al.). Although the pentaplex approach is gaining increasing acceptance for MSI analysis, the present inventors recognize that this approach has certain limitations that have implications in terms of clinical usefulness of this method for determining MSI.

(i) Although the 5 mononucleotide repeat markers in pentaplex PCR are very sensitive and specific in identifying MSI signatures that arise in the setting of MMR-deficiency caused by loss of MLH1, MSH2 and PMS2 proteins, this method only detects ˜50% of all MSH6-defective CRCs. Hence, there is an apparent need for a better marker than can detect all MSH6-deficient

CRCs. Though, a recent article by You et al. claims that the pentaplex PCR could reliably identify all MSH6-deficient CRCs it is still debatable whether a pentaplex PCR can identify all of the MSI cancers.

(ii) The inventors realized that even using 3 of the 5 markers in the pentaplex PCR resulted in comparable sensitivity and specificity. This suggests that a reduced marker panel may be economically more viable and attractive in clinical settings for MSI analysis.

In order to overcome some of the issues presented hereinabove, the inventors used the tetraplex PCR of the present invention to obtain a better MSI marker panel to specifically identify MSH6-deficient CRCs. In this regard, the inventors selected a T25 mononucleotide marker in the 3′untranslated region of the CASP2 gene (CAT25) that displayed a quasi-monomorphic repeat pattern in normal tissue of 200 unrelated individuals of Caucasian origin has been shown to be promising for the identification of Lynch syndrome CRCs. Another unique distinguishing feature of CAT25 marker is that unlike some of the BAT and NR series of markers that demonstrate some degree of polymorphism in a small proportion of African populations, CAT25 has been shown to be highly monomorphic even in individuals of African and Asian origin. However, none of the studies prior to the present invention have thus far addressed the usefulness of CAT25 in the identification of MSH6-deficient CRCs.

The inventors carried out MSI analysis was carried out using FAM-labeled CAT25 mononucleotide marker in a subset of 12 CRCs with germline mutations in the MSH6 gene. Secondly, the inventors developed a novel tetraplex PCR assay which included 3 of the original pentaplex markers (BAT26, NR21 and NR27), along with CAT25 marker. The performance of the newly developed tetraplex PCR and the original pentaplex PCR in a subset of 36MMR-deficient (including 12 cases each for MLH1, MSH2 and MSH6 deficiency), and 36 MMR-proficient CRCs was also evaluated.

Findings of the studies described herein revealed that the CAT25 marker by itself was able to detect frameshift mutations (or MSI) in all 12 MSH6-deficient CRCs. More importantly, comparison efficiency of the pentaplex and tetraplex PCR revealed that while pentaplex PCR was able to detect MSI in all MLH1 and MSH2-defective cancers, it failed to detect MSI in 4 of the 12 MSH6-deficient tumors. In contrast, the newly developed tetraplex PCR demonstrated 100% sensitivity and specificity for MSI analysis, whereby, it was able to detect MSI in all 36 MMR-deficient CRCs, including all 12 MSH6-mutated patients.

The CAT25 described herein is a very sensitive marker for measuring MSI, and is highly specific for detecting MSH6-deficient CRCs. An optimized tetraplex PCR that includes BAT26, NR21, NR27 and CAT25 markers, offers a facile, robust, less expensive (compared to the original pentaplex assay), highly sensitive, and specific assay for the identification of MSI in CRCs.

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Publication No. 20090317815: Mononucleotide Repeats Microsatellite Markers for Detecting Microsatellite Instability.
U.S. Pat. No. 7,662,595: Compounds and Methods for Assessment of Microsatellite Instability (MSI) Status.
Goel, A., Nagasaka, T., Hamelin, R. and Boland, C. R. An Optimized Pentaplex PCR for Detecting DNA Mismatch Repair-Deficient Colorectal Cancers, PLoS ONE, 2010, 5(2), e9393.
You, J-F., Buhard, O., Ligtenberg, M. J. L., Kets, C. M., Niessen, R. C., Hofstra, R. M. W., Wagner, A., Dinjens, W. N. M., Colas, C., Lascols, O., A Collura1,2, Flejou, J-F., Duval, A. and Hamelin, R. Tumours with Loss of MSH6 Expression Are MSI-H When Screened with A Pentaplex of Five Mononucleotide Repeats, British Journal of Cancer, 2010, 103, 1840-1845.

What is claimed is:

1. A system for detecting microsatellite instability (MSI) in a biological sample from a human or animal subject comprising a sufficient number of mononucleotide markers to allow for a detection of a change in a MSH6 gene expression, MSH6 protein expression, or both, wherein the mononucleotide markers comprise CAT25, BAT25, BAT26, NR21, NR22, NR24, NR27, and any combinations thereof.

2. The system of claim 1, wherein the biological sample is a sample isolated from a tumor selected from the group consisting of colorectal cancers, ovarian cancer, urothelial cancers, colorectal tract tumors, colorectal carcinoma, colorectal adenoma, colorectal polyps, gastrointestinal tract tumors, small intestine carcinomas, small intestine polyps, endometrial tumors, endometrial carcinoma, endometrial hyperplasia, and any combinations thereof.

3. The system of claim 2, wherein the tumor is an MSH6-deficient colorectal cancer.

4. The system of claim 1, wherein the system comprises CAT25, BAT26, NR21, and NR27.

5. The system of claim 1, wherein the biological sample is selected from the group consisting of a tissue sample, a fecal sample, a cell homogenate, and one or more biological fluids.

6. A method for detecting one or more tumors caused by microsatellite instability (MSI) in a human or animal subject comprising the steps of:
obtaining a biological sample from the human or the animal subject, wherein the tissue or the biological sample is a sample isolated from a tumor selected from the group consisting of colorectal cancers, ovarian cancer, urothelial cancers, colorectal tract tumors, colorectal carcinoma, colorectal adenoma, colorectal polyps, gastrointestinal tract tumors, small intestine carcinomas, small intestine polyps, endometrial tumors, endometrial carcinoma, endometrial hyperplasia, and any combinations thereof;
isolating a DNA from the biological sample;
amplifying the DNA in a PCR system comprising a sufficient number of mononucleotide markers to allow for a detection of a change in a MSH6 gene expression, MSH6 protein expression, or both, wherein the mononucleotide markers comprise CAT25, BAT25, BAT26, NR21, NR22, NR24, NR27, and any combinations thereof; and
detecting the presence of one or more tumors caused by MSI based on an expression of one or more mismatch repair (MMR) genes, wherein the MMR genes are selected from the group consisting of MLH1, MSH2, MSH3, MSH6, and PMS2.

7. The method of claim 6, further comprising the optional steps of:
obtaining a biological sample from a healthy human or animal subject, wherein the healthy human or animal subject is further defined as a subject not suffering from one or more tumors caused by MSI ;
isolating the DNA from the biological sample of the healthy human or animal subject;
amplifying the DNA in the PCR system; and
comparing the expression of the one or more MMR genes in the biological sample of the healthy human or animal subject with the expression in the biological sample of the human or animal subject suspected of having the one or more tumors caused by MSI.

8. The method of claim 6, wherein the biological sample is selected from the group consisting of a tissue sample, a fecal sample, a cell homogenate, and one or more biological fluids.

9. The method of claim 6, wherein a decrease or an inactivation in the expression of the one or more MMR genes is indicative of the one or more tumors caused by MSI.

10. The method of claim 6, wherein the tumor is a MSH6-deficient colorectal cancer.

11. The method of claim 6, wherein the PCR system comprises CAT25, BAT26, NR21, and NR27.

12. A polymerase chain reaction (PCR) system for detection of a MSH6-deficient colorectal cancer (CRC) comprising:
a CAT25 mononucleotide marker; and
at least one other marker selected from the group consisting of BAT25, BAT26, NR21, NR22, NR24, NR27.

13. The PCR system of claim 12, comprising CAT25, BAT26, NR21, and NR27.

14. The PCR system of claim 12, wherein the system is capable of detecting 85-100% of all MSH6-deficient CRCs.

15. The PCR system of claim 12, wherein the system detects 85%, 86%, 87%, 88%, 89%, 90%, 91%, 92%, 93%, 94%, 95%, 96%, 97%, 98%, 99%, and 100% of all MSH6-deficient CRCs.

16. A method for detecting MSH6-deficient colorectal cancers (CRCs) in a human subject comprising the steps of:
identifying the human subject suspected of having the MSH6-deficient CRC;
obtaining a tissue sample from the human subject;
isolating a DNA from the tissue sample;
amplifying the DNA in a PCR system comprising:
a CAT25 mononucleotide marker; and
at least one other marker selected from the group consisting of BAT25, BAT26, NR21, NR22, NR24, NR27;
detecting the presence of the MSH6-deficient CRC based on a reduced expression or an inactivation of the MSH6 gene, MSH6 protein on, or both in the tissue sample of the human subject suspected of having the MSH6-deficient CRC.

17. The method of claim 16, further comprising the optional steps of:
obtaining a tissue sample from a healthy human subject, wherein the healthy human subject is further defined as a subject not suffering from one or more tumors caused by microsatellite instability (MSI);
isolating the DNA from the tissue sample of the healthy human subject;
amplifying the DNA in the PCR system; and
comparing a MSH6 gene expression, MSH6 protein expression or both in the tissue sample of the healthy human subject with the MSH6 gene expression, MSH6 protein expression, or both in the tissue sample of the human subject suspected of having the MSH6-deficient CRC.

18. The method of claim 16, wherein the PCR system comprises CAT25, BAT26, NR21, and NR27.

19. The method of claim 16, wherein the system is capable of detecting 85-100% of all MSH6-deficient CRCs.

20. The method of claim 16, wherein the system detects 85%, 86%, 87%, 88%, 89%, 90%, 91%, 92%, 93%, 94%, 95%, 96%, 97%, 98%, 99%, and 100% of all MSH6-deficient CRCs.

21. A tetraplex PCR system for detection of MSH6-deficient colorectal cancer (CRC) comprising a CAT25 mononucleotide marker, a BAT26 mononucleotide marker, a NR21 mononucleotide marker, and a NR27 mononucleotide marker.

22. The PCR system of claim 21, wherein the system is capable of detecting 85-100% of all MSH6-deficient CRCs.

23. A method for detecting MSH6-deficient colorectal cancers (CRCs) in a human subject comprising the steps of:
identifying the human subject suspected of having the MSH6-deficient CRC;
obtaining a tissue sample from the human subject;
isolating a genomic DNA from the tissue sample;
amplifying the genomic DNA in a tetraplex PCR system comprising a CAT25 mononucleotide marker, a BAT26 mononucleotide marker, a NR21 mononucleotide marker and a NR27 mononucleotide marker; and
detecting the presence of the MSH6-deficient CRC based on a reduced expression or an inactivation of the MSH6 gene, protein, or both in the tissue or the biological sample of the human subject suspected of having the MSH6-deficient CRC.

24. The method of claim 23, further comprising the optional steps of:
obtaining a tissue sample from a healthy human subject, wherein the healthy human subject is further defined as a subject not suffering from one or more tumors caused by microsatellite instability (MSI);
isolating a genomic DNA from the tissue sample of the healthy human subject;
amplifying the genomic DNA in the PCR system; and
comparing a MSH6 gene expression, MSH6 protein expression, or both in the tissue sample of the healthy human subject with the MSH6 gene expression, MSH6 protein expression, or both in the tissue or the biological sample of the human subject suspected of having the MSH6-deficient CRC.

25. The method of claim 23, wherein the system is capable of detecting 85-100% of all MSH6-deficient CRCs.

26. A kit for the detection of a microsatellite instability (MSI) in a tissue or a biological sample of a human subject suspected of having colorectal cancer (CRC) comprising:
a CAT25 mononucleotide marker;
at least one other marker selected from the group consisting of BAT25, BAT26, NR21, NR22, NR24, NR27; and instructions for use of the kit in the detection of the MSI in the tissue or the biological sample of the human subject suspected of having the colorectal cancer.

27. The kit of claim 26, wherein the kit detects a decreased expression, an inactivation, or both of one or more mismatch repair (MMR) genes in the tissue or the biological sample of the human subject, wherein the MMR genes comprise MLH1, MSH2, MSH3, MSH6, and PMS2.

28. The kit of claim 27, wherein the MMR gene is MSH6.

29. The kit of claim 28, wherein the kit is capable of detecting 85-100% of all MSH6-deficient CRCs.

30. The kit of claim 26, wherein the kit comprises CAT25, BAT26, NR21, and NR27.

31. A kit comprising one or more pairs of primers for amplifying at least four microsatellite loci selected from the group consisting of CAT25, BAT25, BAT26, NR21, NR22, NR24, NR27, and any combinations thereof.

32. The kit of claim 31, wherein the one or more pairs of primers amplify CAT25, BAT26, NR21, and NR27.

33. The kit of claim 31, wherein the kit is adapted for use in the detection of a microsatellite instability (MSI) colorectal cancer (CRC) from a tissue or a biological sample of a human subject, wherein the CRC is a MSH6-deficient CRC.

34. A kit for detecting MSH6-deficient colorectal cancer (CRC) in a human subject comprising one or more pairs of primers for amplifying four microsatellite loci comprising CAT25, BAT26, NR21, and NR27.

35. A system for detecting microsatellite instability (MSI) in a biological sample from a human or animal subject consisting essentially of at least four mononucleotide markers, wherein the mononucleotide markers comprise CAT25, BAT25, BAT26, NR21, NR22, NR24, NR27, and any combinations thereof, wherein the mononucleotide markers are capable of detecting a change in a MSH6 gene expression, MSH6 protein expression, or both.

36. The system of claim 35, wherein the biological sample is a sample isolated from a tumor selected from the group consisting of colorectal cancers, ovarian cancer, urothelial cancers, colorectal tract tumors, colorectal carcinoma, colorectal adenoma, colorectal polyps, gastrointestinal tract tumors, small intestine carcinomas, small intestine polyps, endometrial tumors, endometrial carcinoma, endometrial hyperplasia, and any combinations thereof.

37. The system of claim 36, wherein the tumor is an MSH6-deficient colorectal cancer.

38. The system of claim 35, wherein the system detects CAT25, BAT26, NR21, and NR27.

39. The system of claim 35, wherein the system comprises detection using polymerase chain reaction (PCR).

40. The system of claim 35, wherein the biological sample is selected from the group consisting of a tissue sample, a fecal sample, a cell homogenate, and one or more biological fluids.
